(19) United States
(12) Patent Application Publication
(10) Pub. No.: US 2010/0209368 A1
(43) Pub. Date: Aug. 19, 2010

(54) METHOD AND PREPARATION FOR TREATING BALDNESS

(76) Inventor: Joseph F. Walsh, Sequim, WA (US)

Correspondence Address: OLYMPIC PATENT WORKS PLLC, P.O. BOX 4277, SEATTLE, WA, 98104, US

(21) Appl. No.: 12/703,932

(22) Filed: Feb. 11, 2010

Related U.S. Application Data
(60) Provisional application No. 61/207,420, filed on Feb. 11, 2009.

(51) Int. Cl. A61K 31/327; A61K 31/555; A61K 31/496; A61K 33/04; A61P 31/04; A61P 31/10; A61P 39/06

(52) U.S. Cl. 424/59; 514/714; 514/188; 514/254.07; 424/702

(57) ABSTRACT

Various embodiments of the present invention are directed to hair-loss, and include treatments and preparations. Embodiments of the present invention include bactericides, combinations of bactericides and fungicides, combination of bactericides and vasodilators, and combinations of bactericides, fungicides, and vasodilators that are delivered topically to pilosebaceous units within the scalps of persons suffering from hair loss. The treatment kills or controls microbes that disrupt hair growth by changing, inhibiting, or interrupting one or more biological functions of the pilosebaceous units. Certain embodiments of the present invention contain additional active and inactive ingredients, including anti-inflammatory agents, carriers, emulsifiers, antioxidants, and other such substances.

### CROSS REFERENCE TO RELATED APPLICATION

This application claims benefit from U.S. Provisional Application No. 60/852,247, filed Oct. 17, 2006.

### TECHNICAL FIELD

Embodiments of the present invention are related to treating hair loss.

### BACKGROUND

Although human hair seems most often viewed in terms of personal appearance and social status, human hair does provide many biological and physiological benefits, including protecting the skull and brain from injury, facilitating regulation of skull and brain temperature, and providing protection from UV radiation. Hair, stratum corneum, which is a layer of dead skin above living skin tissue of the scalp, and sebum, which refers to various scalp oils secreted by hair-follicle glands, are humans' first line of defense against UV radiation. UV radiation can damage genetic-information containing biopolymers, including deoxyribonucleic acid (“DNA”), within epidermal cells, potentially leading to melanoma and other cancers. Because hair and dead epidermal cells do not contain functional DNA, cannot undergo cell division, but absorb UV radiation, they provide good protection from the damaging effects of UV radiation.

The social aspects of hair often override the physiological benefits of hair for many people. People with thinning hair, or with significant hair loss referred to as “baldness,” particularly women, are often negatively regarded as less youthful and less attractive. Even mild psychological effects of baldness, such as diminished self-confidence, can have dramatic long term effects on a person's social, home and business lives.

Many treatments for baldness have been sold over the years. These treatments were often sold with misleading or fraudulent claims. The situation became so serious that the Federal Drug Administration (“FDA”) intervened in the marketplace for hair-loss treatments, and now requires that a hair-loss-treatment product successfully undergo clinical trials and be approved by the FDA before it can be sold as a treatment or cure for baldness. To date, two products, minoxidil and finasteride, have been approved by the FDA for treatment of baldness. However, these products provide only a very modest, relatively short-term improvement for most users. Those who experience various degrees of hair loss, medical, pharmaceutical, and hair-products researchers and product developers, hair-care professionals, and many others continue to seek effective methods and preparations for treating hair loss.

### SUMMARY

Various embodiments of the present invention are directed to hair-loss, and include treatments and preparations. Embodiments of the present invention include bactericides, combinations of bactericides and fungicides, combination of bactericides and vasodilators, and combinations of bactericides, fungicides, and vasodilators that are delivered topically to pilosebaceous units within the scalps of persons suffering from hair loss. The treatment kills or controls microbes that disrupt hair growth by changing, inhibiting, or interrupting one or more biological functions of the pilosebaceous units. Certain embodiments of the present invention contain additional active and inactive ingredients, including anti-inflammatory agents, carriers, emulsifiers, antioxidants, and other such substances.

### BRIEF DESCRIPTION OF THE DRAWINGS

FIG. 1 illustrates the Norwood scale for assigning numerical values to represent the stage or degree of baldness in an individual.

FIG. 2 is a diagram of the pilosebaceous unit.

FIG. 3 provides an illustration of the hair-growth cycle.

FIG. 4A shows the chemical structure of benzoyl peroxide.

FIG. 4B shows the chemical structure for zinc pyrithione.

### DETAILED DESCRIPTION

Baldness is the lack of hair growth on the head, where hair once grew. Approximately 25% of men begin experiencing hair loss by age 30, increasing to about two-thirds of men who experience hair loss by age 60. Women experience increasing incidence of frontal hair loss with age. About 57% of women have frontal hair loss by age 80.

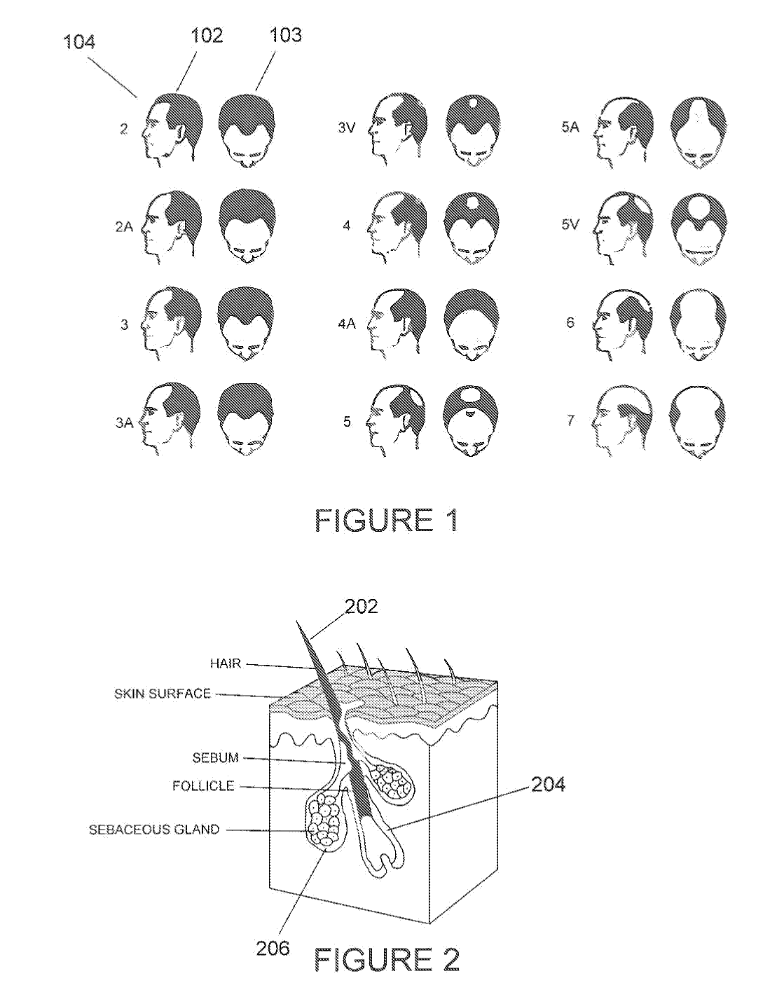

The most common form of baldness is a progressive thinning of the hair in men, called “male pattern baldness” (“MPB”). MPB starts as hair thinning on the lateral sides of the forehead and thinning on the top hack of the head. The thinning areas expand over time and meet as one large bald area surrounded by a horse-shoe-shaped area of hair on the sides and back of head. For research purposes, stages of MPB are classified according to the Norwood Scale. FIG. 1 illustrates the Norwood scale for assigning numerical values to represent the stage or degree of baldness in an individual. In FIG. 1, the stages of baldness corresponding to a particular Norwood numerical value are illustrated by a pair of depictions of the extent of hair loss, such as the pair of depictions 102-103 associated with Norwood Scale value 2 (104 in FIG. 1). Female pattern baldness (“FPB”) is usually found in older women. However, FPB can also occur in teenage girls. Approximately 30% to 40% of women experience FPB in their lifetimes. FPB is associated with genetic factors similar to those associated with MPB, suggesting that it is a type of AGA. The general term for common baldness for both men and women is “androgenic alopecia” (“AGA”).

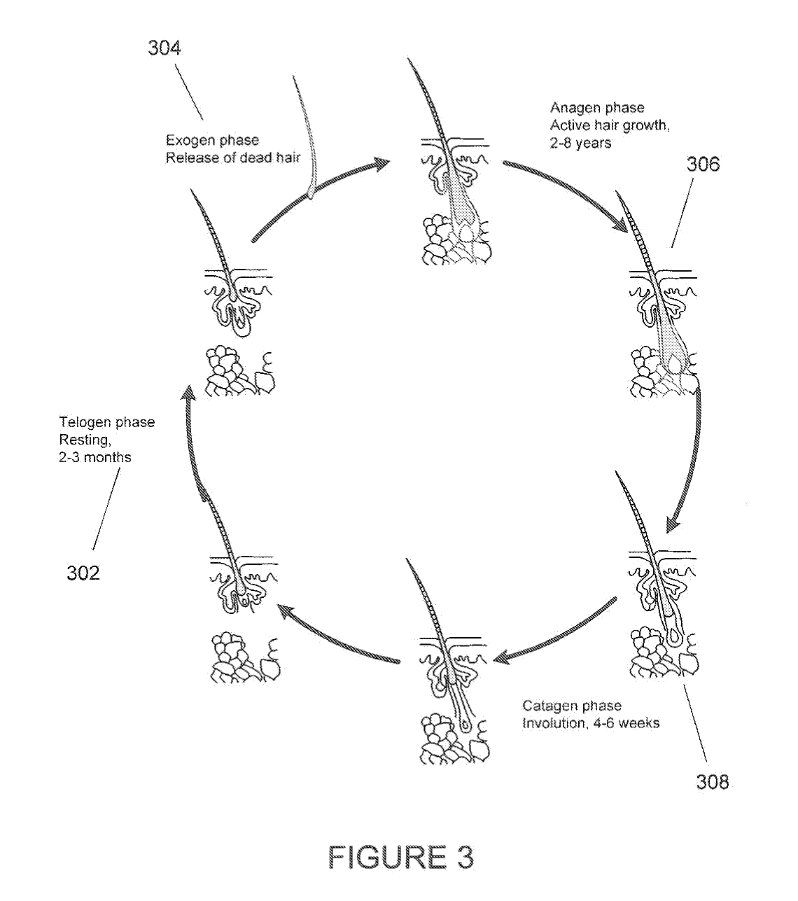

FIG. 2 is a diagram of the pilosebaceous unit. Each of the thousands of pilosebaceous unit within a normal scalp produces a single hair. The pilosebaceous unit includes a hair shaft 202, a hair follicle 204, sebaceous glands which makes sebum 206, and an erector pili muscle which causes the hair to stand up when it contracts. The Sebaceous glands, located in pilosebaceous units, secrete sebum oil, which forms a protective layer of oil on the scalp. The sebum oil layer helps protect the pilosebaceous from attack by microorganisms. On average, about 100,000 hair follicles develop in the pre-natal scalp. Researchers currently believe that no hair follicles are created or lost after birth. Once developed, the follicles undergo a cycle of renewal in three phases. FIG. 3 provides an illustration of the hair-growth cycle. Phases of hair growth include: (1) a telogen phase 302, in which the hair follicle is resting, or dormant; (2) an exogen phase 304, in which a hair is released from the pilosebaceous unit as a new hair begins to grow within the hair follicle; (3) an anagen phase 306, characterized by active hair growth; and (4) a catagen phase 308, in which the hair follicle diminishes in size and regresses. When the number of hairs on an individual's scalp entering the catagen phase exceeds the number of hairs entering the anagen phase, the individual's hair is said to be thinning.

There are as many theories on the causes of baldness. It is generally agreed that baldness is governed by hereditary factors. However, there is much disagreement as to how those hereditary factors contribute to baldness. Testosterone and dehydroeplandrosterone (“DHEA”) are transformed into dihydrotestosterone (“DHT”) by 5-alpha reductase enzymes and hydroxysteroid-hydrogenase-isomerase enzymes in sebaceous glands of the pilosebaceous unit from which hair grows. DHT is a powerful sex hormone as well as a body and facial hair growth promoter, but can also have adverse affects on hair growth. One theory is that DHT may block hair receptors from receiving nutrients. Animal studies support the theory that testosterone and DHT are the main causes of AGA.

Certain studies have implicated yeast as a contributor or cause of baldness. One study concluded that Pityrosporum ovale (“Malassezia”), often present on the scalp of balding individuals, may contribute to baldness in both men and woman. The majority (89.92%) of balding subjects tested positive for Pityrosporum ovale scalp infection, while only 9.52% of non-balding subjects tested positive for Pityrosporum ovale scalp infection.

The FDA has approved two preparations for treating baldness, minoxidil (“Rogain”) and finasteride (“Propecia. Minoxidil is a vasodilator, and is thought to stimulate hair growth by increasing blood flow and hence nutrients to hair roots. Finasteride is a 5-alpha reductase II inhibitor, and is thought to decrease conversion of dihydrotestosterone to DHA. Both provide modest, generally short-term increase in hair count within certain regions of the scalp, but neither comes close to reversing baldness, particularly the numerically larger Norwood-Scale stages of baldness. Researchers have tested the effectiveness of different combinations of minoxidil and pyrithione zinc, a fungicide, on hair density of men with MPB. A 6-month study of 200 patients tested various combinations of minoxidil and pyrithione zinc and found that hair counts were modestly and sustainably improved with daily use of a 1% pyrithione zinc shampoo. However, the benefit of 1% pyrithione-zinc shampoo, used alone, were not statistically significant.

Although the above-discussed research indicates that killing Pityrosporum ovale yeast on the surface of the skin does not significantly reverse baldness, Pityrosporum ovale yeast still may contribute to, or facilitate, AGA. The Pityrosporum ovale yeast may invade the pilosebaceous unit and disrupt the hair growth cycle. The Pityrosporum ovale yeast may also facilitate invasion of the pilosebaceous unit by one or more additional microorganisms, which in turn, disrupt the hair growth cycle in one or more ways, including: (1) secreting toxic wastes that damage hair; (2) disrupting DHT synthesis, binding of DHT to receptors, DHT catabolism, and other DHT-related processes; (3) disrupting hair follicles from receiving nutrients; (4) stimulating pilosebaceous units to produce hormones that signal their local and other, remote hair follicles to enter the resting cycle and/or fail to enter the anagen phase; (5) disrupting pilosebaceous-unit components of the immune system; (6) causing inflammation; and (7) digesting the oily, protective, sebum layer and leaving the pilosebaceous unit more exposed to microorganisms and other deleterious agents and conditions.

Embodiments of the present invention are motivated by a realization that the genetic factors that cause AGA are related to a person's genetically-controlled immune system's ability to fight off microorganisms that disrupt the pilosebaceous units' ability to grow hair. The invading microorganisms may first attack the scalp and then enter the pilosebaceous units, may enter the blood stream through the scalp and then enter pilosebaceous units, or may gain entry to the pilosebaceous by various other means. Invading microorganisms may include Pityrosporum ovale, Propionibacterium acne, bacteria that are a major cause of acne and other skin diseases, and many other fungi, bacteria, and perhaps additional types of microorganisms. Many types of microorganisms reproduce every few hours and are, therefore highly adaptive to many types of pharmaceutical treatments. Accordingly, certain embodiments of the present invention are directed to delivering safe and effective antimicrobials into the tiny pilosebaceous units of the scalp in order to treat the underlying microbial agents responsible for baldness and other hair-loss disorders.

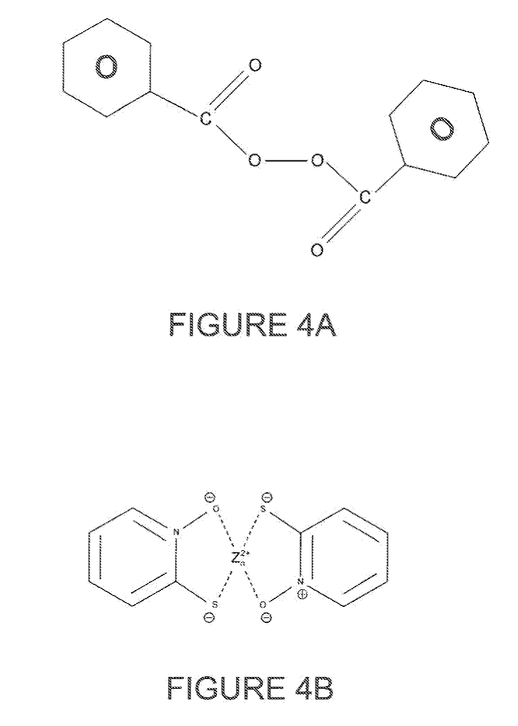

Benzoyl peroxide is an effective bactericide that is used in acne treatments. FIG. 4A shows the chemical structure for benzoyl peroxide. The —O—O— bond is benzoyl peroxide is labile, and cleavage of this bond generates benzyol free radicals that damage bacterial biopolymers. FIG. 4 shows the structure of benzoyl peroxide. Benzoyl peroxide is unstable and has low solubility in water and only slight solubility in ethanol. Benzoyl peroxide is soluble in polyethylene glycol (“PEG”) and isopropyl myristate, but it is not stable in these pure solvents. The stability of benzoyl peroxide in PEG or isopropyl myristate is greatly increased upon the addition of water or ethanol.

The antibiotics erythromycin and tetracycline are known to kill or control Propionibacterium acne and other bacteria. Additional bactericidal agents include: (1) various classes of antibiotic drugs, such as the beta-lactam antibiotics, penicillin and its derivatives, cephalosporins, monobactams, carbapenems, vanomycins, deptomycins, fluoroquinolones, metronidazoles, nitrofurantions, cotrimoxazoles, and aminoglycosidic antibiotics; small-molecule organic compounds, including ethanol, 1-propanol, 2-propanol, and other alcohols, chlorhexidine, brilliant green, chlorhexidine gluconate, phenol, and terpenes; (3) quaternary ammonium compounds, including benzalkonium chloride, cetyl trimethylammonium bromide, cetylpyridinium chloride, and benzethonium chloride; (4) inorganic compounds, including boric acid, hydrogen peroxide, iodine, sodium hypochlorite, and calcium hyopochlorite; and (5) heavy-metal compounds, such as mercurochrome.

Zinc pyrithione is an effective fungicide, as well as antibacterial agent, used in various cosmetic and health-care products. FIG. 4B shows the chemical structure for zinc pyrithione. Zinc pyrthrione is sparingly soluble in water. It can be made soluble in water by adding an amine, such as ethanolamine, or by adding an alcohol, such as ethanol, in combination with a chelating agent, such as ethylenediaminetetraacetic acid (“EDTA”). Other antifungal agents include: (1) polyene antifungals, including natamycin, filipin, nystatin, amphotericin B, and candicin; (2) imidazole and triazole antifungals, including miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, and terconazole: (3) allylamines, including terbinafine, amorolfine, naftifine, and butenafine; (4) echinocandins, including anidulafungin, caspofungin, and micafungin; and (5) other antifungals, including benzoic acid, ciclopirox, tolnaftate, undecylenic acid, flucytosine, griseofulvin, haloprogin, allicin, tea tree oil, citronella oil, lemon grass, orange oil, palmarosa oil, patchouli, lemon myrtle, neem seed oil, coconut oil, zinc, selenium, and pentamidine dapsone atovaquone.

Certain embodiments of the present invention include a bactericidal agent, combined with additional ingredients that can be topically applied to the scalp to introduce a sufficient quantity of the bactericidal agent into pilosebaceous units in order to kill or inhibit growth of microorganisms responsible for hair loss. Additional embodiments of the present invention include a bactericidal agent and an antifungal agent, combined with additional ingredients, that can be topically applied to the scalp to introduce a sufficient quantity of the bactericidal and antifungal agents to the scalp and sufficient quantity of the bactericidal agent into pilosebaceous units in order to kill or inhibit growth of microorganisms responsible for hair loss, and that additionally kill or inhibit yeast and other fungi that contribute to hair loss. Additional embodiments of the present invention include a bactericidal agent and an antifungal agent, combined with additional ingredients, that can be topically applied to the scalp to introduce a sufficient quantity of the bactericidal and antifungal agents into pilosebaceous units in order to kill or inhibit growth of microorganisms responsible for hair loss, and that additionally kill or inhibit yeast and other fungi that contribute to hair loss. Additional embodiments of the present invention include a broadband agent that is both a bactericidal and an antifungal agent, combined with additional ingredients, that can be topically applied to the scalp to introduce a sufficient quantity of this bactericidal and antifungal agent into pilosebaceous units in order to kill or inhibit growth of microorganisms responsible for hair loss. Additional embodiments of the present invention include a bactericidal agent, an antifungal agent, and a vasodilator combined with additional ingredients, that can be topically applied to the scalp to introduce a sufficient quantity of the bactericidal agent into pilosebaceous units in order to kill or inhibit growth of microorganisms responsible for hair loss, additionally kill or inhibit yeast and other fungi that contribute to hair loss, and promote increased delivery of nutrients to hair follicles.

In one family of embodiments of the present invention, the bactericidal agent, bactericidal agent and antifungal agent, or bactericidal agent, antifungal agent, and vasodilator are combined with additional ingredients to create a shampoo that can be applied multiple times per day, on a daily basis, or at longer intervals, over the course of between several months and years, to kill or inhibit microorganisms within pilosebaceous units and restore hair growth to bald or thinning regions of a user's scalp. The additional ingredients may include water and other carriers and co-solvents, one or more surfactants, such as sodium lauryl sulfate, sodium laureth sulfate, and cocamidopropyl betaine, and other surfactants, inorganic salts, preservatives, fragrances, emulsifiers, such as polyethylene glycol, antioxidants, and other such ingredients. A shampoo is, in many ways, an ideal vehicle for treating hair loss according to embodiments of the present invention. Shampoo is commonly and regularly used, for example. Treatments based on shampoos thus do not require a user to adapt to a new procedure and incur inconveniences and lifestyle changes. Another family of embodiments of the present invention include the bactericidal agent, bactericidal agent and antifungal agent, or bactericidal agent, antifungal agent, and vasodilator are combined with additional ingredients to create a leave-on solution or lotion that can be applied multiple times per day, on a daily basis, or at longer intervals, over the course of between several months and years, to kill or inhibit microorganisms within pilosebaceous units and restore hair growth to bald or thinning regions of a user's scalp. The additional ingredients may include water and other carriers and co-solvents, inorganic salts, preservatives, fragrances, emulsifiers, such as polyethylene glycol, antioxidants, and other such ingredients.

One embodiments of the present invention is a hair-loss-treatment shampoo that contains, as one active ingredient, the antibacterial agent benzoyl peroxide at a volume percent of between 0.1 and 10. Another embodiment of the present invention is a hair-loss-treatment leave-on solution or lotion that contains, as one active ingredient, the antibacterial agent benzoyl peroxide at a volume percent of between 0.1 and 10. Another embodiment of the present invention is a hair-loss-treatment shampoo that contains, as active ingredients, the antibacterial agent benzoyl peroxide at a volume percent of between 0.1 and 5.0 and the antifungal compound zinc pyrithione at a volume percent of between 0.1 and 3.0. Another embodiment of the present invention is a hair-loss-treatment leave-on solution or lotion that contains, as active ingredients, the antibacterial agent benzoyl peroxide at a volume percent of between 0.1 and 5.0 and the antifungal compound zinc pyrithione at a volume percent of between 0.1 and 3.0. Yet another embodiment of the present invention is a hair-loss-treatment shampoo that contains, as active ingredients, the antibacterial agent benzoyl peroxide at a volume percent of between 0.1 and 5.0, the antifungal compound zinc pyrithione at a volume percent of between 0.1 and 3.0, and the vasodilator minoxidil at a volume percent of between 0.1 and 8.0. Yet another embodiment of the present invention is a hair-loss-treatment leave-on solution or lotion that contains, as active ingredients, the antibacterial agent benzoyl peroxide at a volume percent of between 0.1 and 5.0, the antifungal compound zinc pyrithione at a volume percent of between 0.1 and 3.0, and the vasodilator minoxidil at a volume percent of between 0.1 and 8.0. Additional embodiments of the present invention include hair-loss-treatment shampoos and leave-on solutions that contain, as active ingredients, the antibacterial agent benzoyl peroxide at a volume percent of between 1.5 and 3.0, the antifungal compound Zinc pyrithione at a volume percent of between 0.5 and 2.0, and the vasodilator minoxidil at a volume percent of between 0.1 and 8.0. Additional embodiments of the present invention include hair-loss-treatment shampoos and leave-on solutions that contain, as active ingredients, the antibacterial agent benzoyl peroxide at a volume percent of between 2.0 and 2.7, the antifungal compound zinc pyrithione at a volume percent of between 1.0 and 2.0, and the vasodilator minoxidil at a volume percent of between 0.1 and 8.0. Benzoyl peroxide may produce the side effect of exfoliating surface layers of dead skin on the scalp, and producing skin irritation and inflammation. Treatment based on a shampoo solution ameliorates these side effects, since the benzoyl peroxide remains in contact with the skin and hair for a relatively short period of time, and in then rinsed away. Certain leave-on-solution embodiments of the present invention ameliorate these side effects by sequestering the benzoyl peroxide in a gel that slowly releases benzoyl peroxide over time, and thus only exposing the scalp to very small amounts of the benzoyl peroxide at any given time. In addition, UV-blocking and UV-absorbing compounds and anti-inflammatory agents may be additionally added to shampoos and leave-on solutions that represent embodiments of the present invention in order to additionally ameliorate any side effects produced by benzoyl peroxide.

Embodiments of the present invention have been tested, and have shown to restore hair growth in bald areas of scalp stating at about six months following the beginning of daily treatment with a hair-loss-ameliorating shampoo or leave-on solution that represent embodiments of the present invention. The hair-loss-ameliorating shampoo or leave-on solution stimulated hair growth over a wide area of the scalp with very advanced hair loss, as represented by a Norwood Scale, Grade VII. New hair growth appeared in areas of scalp that had been bald for up to forty years.

Although embodiments of the present invention contains both bactericides and anti-fungals, preservatives are added, in certain embodiments of the present invention, to the hair-loss-ameliorating shampoo or leave-on solution or lotion to preserve potency and insure long shelf-life. Example preservatives are propylparaben, methylparaben and diazolidinyl urea. In many embodiments of the present invention, although water is the primary solvent for either the shampoo or leave-on solution, additional co-solvents are added, including PEG, ethanol, isopropyl myristate, and other co-solvents to facilitate solution of bactericides and/or fungicides and to stabilize solvated bactericides and/or fungicides.

Although the present invention has been described in terms of a particular embodiment, it is not intended that the invention be limited to this embodiment. Modifications within the spirit of the invention will be apparent to those skilled in the art. For example, additional embodiments of the present invention include shampoos and leave-on solutions in which a different antibacterial agent is substituted for, or added in combination with, benzoyl peroxide, a different antifungal agent is substituted for, or added in combination with, zinc pyrithione, and a different vasodilator is substituted for, or added in combination with, minoxidil. These different antibacterial agents, antifungal agents, and vasodilators may be selected from among the above-mentioned compounds and substances or may be selected from additional antibacterial agents, antifungal agents, and vasodilators such that, when combined with a solvent and other ingredients of a hair-loss-treatment shampoo or leave-on solution or lotion, an effective concentration of the antibacterial agents, antifungal agents, and/or vasodilators is produced in the pilosebaccous units of the scalp to which the include shampoos and leave-on solutions or lotions are applied.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purposes of illustration and description. They are not intended to be exhaustive of or to limit the invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A hair-loss-ameliorating shampoo comprising:
a solvent;
an antibacterial agent that, when the shampoo is applied to a scalp during hair washing, accumulates at sufficient concentration within the pilosebaceous units, resulting in killing or inhibiting microorganisms that cause or contribute to hair loss; and
a surfactant.

2. The hair-loss-ameliorating shampoo of claim 1
wherein the solvent is water; and
wherein the antibacterial agent is benzoyl peroxide at a volume percent of between 0.1 and 10.0.

3. The hair-loss-ameliorating shampoo of claim 1 wherein the surfactant is one of
sodium lauryl sulfate;
sodium laureth sulfate; and
cocamidopropyl betaine.

4. The hair-loss-ameliorating shampoo of claim 1 further including one or more of:
an inorganic salt;
a preservative;
a fragrance;
a co-solvent;
an emulsifier; and
an antioxidant.

5. The hair-loss-ameliorating shampoo of claim 1 further including one or more of:
a UV-blocking substance;
a UW-absorbing substance; and
an anti-inflammatory agent.

6. A hair-loss-ameliorating shampoo comprising:
a solvent;
an antibacterial agent that, when the shampoo is applied to a scalp during hair washing, accumulates at sufficient concentration within the pilosebaceous units, resulting in killing or inhibiting microorganisms that cause or contribute to hair loss;
an antifungal agent; and
a surfactant.

7. The hair-loss-ameliorating shampoo of claim 6
wherein the solvent is water;
wherein the antibacterial agent is benzoyl peroxide at a volume percent of between 0.1 and 10.0; and
wherein the antifungal agent is zinc pyrithione at a volume percentage of between 0.1 and 3.0.

8. The hair-loss-ameliorating shampoo of claim 6 wherein the surfactant is one of
sodium lauryl sulfate;
sodium laureth sulfate; and
cocamidopropyl betaine.

9. The hair-loss-ameliorating shampoo of claim 6 further including one or more of:
an inorganic salt;
a preservative;
a fragrance;
a co-solvent;
an emulsifier; and
an antioxidant.

10. The hair-loss-ameliorating shampoo of claim 6 further including one or more of:
a UV-blocking substance;
a UW-absorbing substance; and
an anti-inflammatory agent.

11. The hair-loss-ameliorating shampoo of claim 6 wherein the antifungal agent is one or more of:
ketoconazole;
selenium sulfide; and
zinc pyrithione.

12. A hair-loss-ameliorating shampoo comprising:
a solvent;
an antibacterial agent that, when the shampoo is applied to a scalp during hair washing, accumulates at sufficient concentration within the pilosebaceous units, resulting in killing or inhibiting microorganisms that cause or contribute to hair loss;
an antifungal agent;
a vasodilator; and
a surfactant.

13. The hair-loss-ameliorating shampoo of claim 12
wherein the solvent is water;
wherein the antibacterial agent is benzoyl peroxide at a volume percentage of between 0.1 and 10.0;
wherein the antifungal agent is zinc pyrithione at a volume percentage of between 0.1 and 3.0; and
wherein the vasodilator is minoxidil at a volume percentage of between 0.1 and 8.0.

14. The hair-loss-ameliorating shampoo of claim 12 wherein the surfactant is one of
sodium lauryl sulfate;
sodium laureth sulfate; and
cocamidopropyl betaine.

15. The hair-loss-ameliorating shampoo of claim 12 further including one or more of:
an inorganic salt;
a preservative;
a fragrance;
a co-solvent;
an emulsifier; and
an antioxidant.

16. The hair-loss-ameliorating shampoo of claim 12 further including one or more of:
a UV-blocking substance;
a UW-absorbing substance; and
an anti-inflammatory agent.

17. The hair-loss-ameliorating shampoo of claim 12 wherein the antifungal agent is one or more of:
ketoconazole;
selenium sulfide; and
zinc pyrithione.

18. A hair-loss-ameliorating leave-on solution comprising:
a solvent; and
an antibacterial agent that, when the leave-on solution is applied to a scalp, accumulates at sufficient concentration within the pilosebaceous units, resulting in killing or inhibiting microorganisms that cause or contribute to hair loss.

19. The hair-loss-ameliorating leave-on solution of claim 18
wherein the solvent is water; and
wherein the antibacterial agent is benzoyl peroxide at a volume percent of between 0.1 and 10.0.

20. The hair-loss-ameliorating leave-on solution of claim 18 further including one or more of:
an inorganic salt;
a preservative;
a fragrance;
a co-solvent;
an emulsifier; and
an antioxidant.

21. The hair-loss-ameliorating leave-on solution of claim 18 further including one or more of:
a UV-blocking substance;
a UW-absorbing substance; and
an anti-inflammatory agent.

22. A hair-loss-ameliorating leave-on solution comprising:
a solvent;
an antibacterial agent that, when the leave-on solution is applied to a scalp, accumulates at sufficient concentration within the pilosebaceous units, resulting in killing or inhibiting microorganisms that cause or contribute to hair loss;
an antifungal agent; and
a surfactant.

23. The hair-loss-ameliorating leave-on solution of claim 22
wherein the solvent is water;
wherein the antibacterial agent is benzoyl peroxide at a volume percent of between 0.1 and 10.0; and
wherein the antifungal agent is zinc pyrithione at a volume percentage of between 0.1 and 3.0.

24. The hair-loss-ameliorating leave-on solution of claim 22 further including one or more of:
an inorganic salt;
a preservative;
a fragrance;
an emulsifier; and
an antioxidant.

25. The hair-loss-ameliorating leave-on solution of claim 22 further including one or more of:
a UV-blocking substance;
a UW-absorbing substance; and
an anti-inflammatory agent.

26. The hair-loss-ameliorating leave-on solution of claim 22 wherein the antifungal agent is one or more of:
ketoconazole;
selenium sulfide; and
zinc pyrithione.

27. A hair-loss-ameliorating leave-on solution comprising:
a solvent;
an antibacterial agent that, when the leave-on solution is applied to a scalp, accumulating at sufficient concentration within the pilosebaceous units, resulting in killing or inhibiting microorganisms that cause or contribute to hair loss;
an antifungal agent;
a vasodilator; and
a surfactant.

28. The hair-loss-ameliorating leave-on solution of claim 27
wherein the solvent is water;
wherein the antibacterial agent is benzoyl peroxide at a volume percentage of between 0.1 and 10.0;
wherein the antifungal agent is zinc pyrithione at a volume percentage of between 0.1 and 3.0; and
wherein the vasodilator is minoxidil at a volume percentage of between 0.1 and 8.0.

29. The hair-loss-ameliorating leave-on solution of claim 27 further including one or more of:
an inorganic salt;
a preservative;
a fragrance;
a co-solvent;
an emulsifier; and
an antioxidant.

30. The hair-loss-ameliorating leave-on solution of claim 27 further including one or more of:
a UV-blocking substance;
a UW-absorbing substance; and
an anti-inflammatory agent.

31. The hair-loss-ameliorating leave-on solution of claim 26 wherein the antifungal agent is one or more of:
ketoconazole;
selenium sulfide; and
zinc pyrithione.
